(19) United States
(12) Patent Application Publication
(10) Pub. No.: US 2015/0258158 A1
(43) Pub. Date: Sep. 17, 2015

(54) COMPOSITIONS AND METHODS FOR INHIBITION OF TRIGLYCERIDE SYNTHESIS VIA SYNERGISTIC COMBINATION OF BOTANICAL FORMULATIONS

(71) Applicant: Access Business Group International LLC, Ada, MI (US)

(72) Inventors: Rodney A. Velliquette, Ada, MI (US); Russell Keith Randolph, Anaheim, CA (US); Jeffrey D. Scholten, Grand Rapids, MI (US); Arun Rajgopal, Grand Rapids, MI (US); Kevin Gellenbeck, Poway, CA (US); Chun Hu, Irvine, CA (US); Kerry Grann, Lansing, MI (US); Kristin Morris, Grand Rapids, MI (US)

(73) Assignee: Access Business Group International LLC, Ada, MI (US)

(21) Appl. No.: 14/632,477

(22) Filed: Feb. 26, 2015

Related U.S. Application Data
(60) Provisional application No. 61/952,534, filed on Mar. 13, 2014.

(51) Int. Cl. A61K 36/87; A61K 8/97; A61Q 19/00; A61K 36/48

(52) U.S. Cl. CPC A61K 36/87; A61K 36/48; A61K 8/97; A61Q 19/00; A61K 2800/10; A61K 2800/5922

(57) ABSTRACT

Compositions and methods for inhibition of triglyceride synthesis via synergistic combination of botanical extracts are described. Specifically, the present invention relates to treating or preventing weight gain or obesity, promoting weight loss, appetite suppression, or the like, as well as managing skin oil production through a synergistic inhibition of diacylglycerol acyltransferase-I (DGAT-I) enzyme involved in the triglyceride synthesis and modulation of sterol regulatory element binding protein Ic (SREBP-Ic) and/or peroxisome proliferator activated receptor gamma coactivator I-alpha (PGCIα).

### RELATED APPLICATIONS

The present patent document claims the benefit of the filing date under 35 U.S.C. §119(e) of Provisional U.S. Patent Application Ser. No. 61/952,534, filed Mar. 13, 2014, which is hereby incorporated by reference in its entirety.

### BACKGROUND

Compositions and methods for inhibition of triglyceride synthesis via synergistic combination of botanical extracts are described. Specifically, the following description relates to treating or preventing weight gain or obesity, promoting weight loss, appetite suppression, or the like, as well as managing skin oil production through a synergistic inhibition of diacylglycerol acyltransferase-I (DGAT-I) enzyme involved in the triglyceride synthesis and modulation of sterol regulatory element-binding protein Ic (SREBP-Ic) and/or peroxisome proliferator—activated receptor gamma coactivator I-alpha (PGCIα).

DGAT-I is an enzyme that catalyzes the terminal step in mammalian triglyceride (fat) synthesis. DGAT-I functions during absorption and assimilation of dietary fat, and also in the deposition of fat in adipose tissue as well as other tissues, such as, e.g., sebaceous glands in the skin.

SREBP-Ic is a transcription factor that belongs to a basic helix-loop-helix/leucine zipper transcription factor family, which also includes SREBP-Ia and SREBP2 (Brown and Goldstein, 1997, Cell 89, 331-340). Among the SREBP family of transcription factors, SREBP-Ic preferentially regulates genes involved in triglyceride and fatty acid synthesis, whereas SREBP-2 regulates genes related to cholesterol synthesis.

PGCIα is a transcriptional coactivator that mediates many biological programs related to energy metabolism. Originally described as a coactivator of PPARγ that modulated expression of uncoupling protein I (UCPI) and thermogenesis in brown fat, it has also been shown to control mitochondrial biogenesis and oxidative metabolism in many cell types. PGCIα is induced in muscle by exercise and stimulates many of the known beneficial effects of exercise in muscle: mitochondrial biogenesis, angiogenesis and fiber-type switching (Handschin and Spiegelman (2008) Nature 454, 463-469). It also provides resistance to muscular dystrophy and denervation-linked muscular atrophy (Sandri et al. (2006) Proc. Natl. Acad. Sci. USA 103, 16260-16265). The healthful benefits of elevated muscle expression of PGCIα may go beyond the muscle tissue itself. Transgenic mice with mildly elevated muscle PGCIα are dramatically resistant to age-related obesity and diabetes and have a prolonged life-span (Wenz et al. (2009) Proc. Natl. Acad. Sci. USA 106, 20405-20410), which suggests that PGCIα might stimulate the secretion of factors from skeletal muscle that affects the health and function of other tissues and may relate to body weight management.

A class of drugs called bile acid binding resins, e.g., cholestyramine, function to inhibit the absorption of dietary fat by formation of physical complexes with ingested fat, rendering the fat in complex form that is not absorbed by the body. Bile acid binding resins are prescribed for individuals that have difficulties with dietary fat effects on blood lipids and metabolism. However, the physical complexes formed between bile acid binding resins and dietary fat induce steatorrhea, an undesirable side effect.

As such, improved compositions and methods for use in management of body weight as well as management of skin oil production, such as by inhibiting triglyceride synthesis or absorption are desirable.

### SUMMARY

In one embodiment, a composition comprises an effective amount of an extract mixture comprising a whole grape extract and at least one of a honeybush extract and a grape seed extract was found to be effective. In the composition, the at least one of a honeybush extract and a grape seed extract may be present in an amount with respect to the whole grape extract to synergistically inhibit diacylglycerol acyltransferase-I (DGAT-I). In the composition, the honeybush extract may be present in an amount to activate PGCIα promoter activity. In the composition, the grape seed extract may be present in an amount to inhibit SREBPIc promoter activity. The composition may further include a pharmaceutically, nutraceutically or cosmetically acceptable carrier, diluent, or excipient, wherein the extract mixture comprises from about 0.5 weight percent (wt %) to about 90 wt % of the composition. Alternatively, the composition may include a pharmaceutically, nutraceutically or cosmetically acceptable carrier, diluent, or excipient, wherein the extract mixture comprises from about 0.5 weight percent (wt %) to about 75 wt % of active ingredients of the composition. Alternatively, the composition may include a pharmaceutically, nutraceutically or cosmetically acceptable carrier, diluent, or excipient, wherein the extract mixture comprises from about 0.5 weight percent (wt %) to about 50 wt % of active ingredients of the composition. The composition may be for oral administration that may be formulated as a tablet, capsule, powder, or granule. The composition may be for topical administration that may be formulated as a cream, gel, lotion, spray solution, pad, bandage, and a transdermal patch. The composition may modulate dietary fat absorption and assimilation in a subject. The composition may suppress the synthesis of fat in adipose tissue. The composition may inhibit diacylglycerol acyltransferase-I enzyme. The composition may modulate production of skin oil.

Another embodiment provides a food or a drink that includes a composition that includes an effective amount of an extract mixture comprising a whole grape extract and at least one of a honeybush extract and a grape seed extract.

Yet another embodiment relates to a weight management product that includes a composition comprising an effective amount of an extract mixture comprising a whole grape extract and at least one of a honeybush extract and a grape seed extract. The weight management product may be formulated for oral administration. The weight management product may be a food or a drink comprising the composition as an active substance. The weight management product may be a pharmaceutical comprising the composition as an active substance. The weight management product may be formulated for topical administration. The weight management product may be formulated for cosmetic use. The weight management product may include a pharmaceutically, nutraceutically or cosmetically acceptable carrier, diluent, or excipient, wherein the extract mixture comprises from about 0.5 weight percent (wt %) to about 90 wt % of the composition. The weight management product may include a pharmaceutically, nutraceutically or cosmetically acceptable carrier, diluent, or excipient, wherein the extract mixture comprises from about 0.5 weight percent (wt %) to about 75 wt % of the composition. The weight management product may include a pharmaceutically, nutraceutically or cosmetically acceptable carrier, diluent, or excipient, wherein the extract mixture comprises from about 0.5 weight percent (wt %) to about 50 wt % of the composition.

Another embodiment relates to a method for treating, preventing, or managing body weight gain in a subject. The method includes administering to the subject a composition comprising an effective amount of an extract mixture comprising a whole grape extract and at least one of a honeybush extract and a grape seed extract to effectively treat, prevent, or manage weight gain in the subject.

A further embodiment relates to a method of reducing fat absorption, transport, deposition, production and secretion in a subject. The method includes administering to the subject a composition comprising an effective amount of an extract mixture comprising a whole grape extract and at least one of a honeybush extract and a grape seed extract to effectively reduce fat absorption, transport, deposition, production and secretion in the subject.

Another embodiment relates to a method of facilitating body weight lost in a subject. The method includes administering a composition comprising an effective amount of an extract mixture comprising a whole grape extract and at least one of a honeybush extract and a grape seed extract to effectively facilitate weight loss in the subject.

Another embodiment relates to a method of maintaining body weight of a subject. The method includes administering to the subject a composition comprising an effective amount of an extract mixture comprising a whole grape extract and at least one of a honeybush extract and a grape seed extract to effectively maintain body weight of the subject.

Another embodiment relates to a method for treating, preventing or managing skin conditions or disorders associated with skin oil production in a subject. The method includes administering to the subject a composition comprising an effective amount of an extract mixture comprising a whole grape extract and at least one of a honeybush extract and a grape seed extract to effectively treat, prevent or manage skin conditions or disorders associated with skin oil production in the subject.

A further embodiment relates to a method of synergistically inhibiting DGAT-I in a subject. The method includes administering to the subject a composition comprising an effective amount of an extract mixture comprising a whole grape extract and at least one of a honeybush extract and a grape seed extract to synergistically inhibit DGAT-I in the subject.

### BRIEF DESCRIPTION OF THE DRAWINGS

FIG. 1 depicts a graph illustrating a dose response and EC50 value of the whole grape extract on DGAT-I enzyme inhibition in a cell-free assay.

FIG. 2 depicts a graph illustrating the dose response and EC50 value of the whole grape extract on cellular DGAT-I inhibition.

FIG. 3A depicts a graph illustrating the percent change in triglyceride response before and after treatment in a placebo group.

FIG. 3B depicts a graph illustrating the percent change in triglyceride response before and after treatment in whole grape extract group.

FIG. 3C depicts a graph illustrating the percent change in triglyceride response after 7 days of placebo or whole grape extract treatment.

FIG. 4 depicts a bar graph illustrating effect of honeybush extract on cellular PGCIα bioassay.

FIG. 5 depicts a graph illustrating the effect of grape seed extract on cellular SREBPIc promoter activity.

FIG. 6A depicts a bar graph showing percent cellular triglycerides in response to treatment with whole grape extract and grape seed extract (20 μg/mL) as compared to control, whole grape extract alone, grape seed extract alone (20 μg/mL) and a predicted response.

FIG. 6B depicts a bar graph showing percent cellular triglycerides in response to treatment with whole grape extract and grape seed extract (30 μg/mL) as compared to control, whole grape extract alone, grape seed extract alone (30 μg/mL) and a predicted response.

FIG. 6C depicts a bar graph showing percent cellular triglycerides in response to treatment with whole grape extract and grape seed extract (40 μg/mL) as compared to control, whole grape extract alone, grape seed extract alone (40 μg/mL) and a predicted response.

FIG. 7A depicts a bar graph showing percent cellular triglycerides in response to treatment with whole grape extract and honeybush extract (10 μg/mL) as compared to control, whole grape extract alone, honeybush extract alone (10 μg/mL) and a predicted response.

FIG. 7B depicts a bar graph showing percent cellular triglycerides in response to treatment with whole grape extract and honeybush extract (25 μg/mL) as compared to control, whole grape extract alone, honeybush extract alone (25 μg/mL) and a predicted response.

FIG. 7C depicts a bar graph showing percent cellular triglycerides in response to treatment with whole grape extract and honeybush extract (50 μg/mL) as compared to control, whole grape extract alone, honeybush extract alone (50 μg/mL) and a predicted response.

### DETAILED DESCRIPTION

Currently, there are no formulations or commercial products that inhibit fat synthesis via inhibition of DGAT-I and SREBPIc, and activation of PGCIα and that may be useful in body weight management, as well as, skin oil management applications.

Compositions and methods are described for weight management and, more particularly, to compositions that include a whole grape extract in combination with a honeybush extract or a grape seed extract, or both, as well as to methods of use thereof such as for weight management, treating or preventing weight gain or obesity, promoting weight loss, appetite suppression, or the like via synergistically inhibiting the DGAT-I enzyme, as well as inhibition of SREBPIc promoter activity and activation of PGCIalpha promoter activity.

Also, compositions are described for treating, preventing or managing skin conditions or disorders associated with skin oil production in a subject and, more particularly, to compositions that include a whole grape extract in combination with a honeybush extract or a grape seed extract, or both, as well as to methods of use thereof such as for treating, preventing or managing skin conditions or disorders associated with skin oil production in a subject, or the like via synergistically inhibiting the DGAT-I enzyme, as well as inhibition of SREBPIc promoter activity and activation of PGCIalpha promoter activity.

### DEFINITIONS

The term “composition” refers to a product that treats, improves, promotes, increases, manages, controls, maintains, optimizes, modifies, reduces, inhibits, or prevents a particular condition associated with a natural state or biological process. The term composition includes, but is not limited to, pharmaceutical (i.e., drug), cosmetic, food, food ingredient or dietary supplement compositions that include an effective amount of an extract mixture or a component thereof. Exemplary compositions include topical creams and lotions, dietary supplements, beverages and beverage mixes.

The term “pharmaceutical composition” refers to a composition that may be used to produce a medicinal product (i.e., drug), such that it would require a prescription from a physician or veterinarian.

The term “cosmetic composition” refers to a composition that may be used to produce care substances used to enhance the appearance or odor of the human body that may or may not require a prescription from a physician or veterinarian.

The term “food composition” refers to a composition that may be used to produce a food or a beverage product.

The term “dietary supplement” as used herein, refers to a product that improves, promotes, increases, manages, controls, maintains, optimizes, modifies, reduces, inhibits, or prevents a particular condition associated with a natural state or biological process (i.e., are not used to diagnose, treat, mitigate, cure, or prevent disease). For example, with regard to weight-related conditions, dietary supplements may be used to promote weight loss, manage weight gain, maintain weight, reduce caloric intake, increase muscle mass, or the like. For example, with regard to the management of skin oil production related conditions, dietary supplements may be used to promote reduction in the skin oil production, reduction in the occurrence of skin acne, or the like. Exemplary dietary supplements include one or more of a dietary ingredient such as a vitamin, a mineral, an herb or other botanical, an amino acid, or any other substance used to supplement the diet by increasing total dietary intake, or a concentrate, metabolite, constituent, extract, or any combination thereof. In certain embodiments, dietary supplements are a special category of food and are not a drug.

The term “extract mixture” refers to a mixture or a combination of two or more different extracts. The extracts comprising the mixture may be at equivalent or different amounts or ratios.

As used herein, the term “extract” refers to a solid, viscid, or liquid substance or preparation that includes an active ingredient of a substance of plant, such as whole grape, honeybush or grape seed in a concentrated form. The term “extract” is intended to include not only a crude extract produced from whole grape, honeybush and/or grape seed, by use of a solvent selected from among water, lower alcohols of 1 to 4 carbon atoms, such as methanol, ethanol, butanol, etc., ethylene, acetone, hexane, ether, chloroform, ethylacetate, butylacetate, dichloromethane, N,N-dimethylformamide (DMF), dimethylsulfoxide (DMSO), 1,3-butylene glycol, propylene glycol and a combination thereof, but also a fraction of the crude extract in such a solvent. So long as it assures the extraction and preservation of the active ingredient, any extraction method may be employed. Examples of the extraction methods include solvent extraction, superficial fluid extraction, etc. The fraction may include those obtained by partitioning the crude extract between two solvents which have different polarities and eluates obtained by eluting the crude extract loaded into a silica gel-filled column using a hydrophobic solvent, a hydrophilic solvent or a combination thereof as a mobile phase. In addition, the extracts may be in a concentrated liquid phase or a solid phase as a result of removing the extraction solvent by freeze drying, vacuum drying, hot-air drying, or spray drying. Preferably, the extracts may be crude extracts produced from whole grape, honeybush or grape seed using a solvent selected from the group consisting of water, ethanol and a combination thereof, or a fraction of the crude extract.

As used herein, the term “effective amount” or “therapeutically effective amount” of a composition, extract mixture, component of the extract mixture, and/or active agent or ingredient refers to an amount effective at dosages and for periods of time sufficient to achieve a desired result. For example, the “effective amount” or “therapeutically effective amount” refers to that amount of a composition, extract mixture, component of the extract mixture, and/or active agent or ingredient of this invention which, when administered to a subject (e.g., mammal, such as a human), is sufficient to effect treatment, including any one or more of: (1) synergistically inhibiting DGAT-I in a subject; (2) treating, preventing or managing weight gain in a subject; (3) facilitating weight loss; (4) suppressing appetite in a subject; (5) treating or preventing obesity in a subject; (6) modifying fat uptake in a subject (e.g., by reducing fat absorption, transport, deposition, production and/or secretion); (7) reducing absorption, assimilation and deposition of dietary fat by inhibiting the synthesis of fat that occurs within intestinal cells; (8) interfering with increasing metabolism to promote weight loss or prevent weight gain in a subject; (9) maintaining body weight of the mammal; (10) treating, preventing, or managing skin condition or disorder associated with skin oil production in a subject. The amount of a composition, extract mixture, component of the extract mixture, and/or active agent or ingredient of this disclosure that constitutes a “therapeutically effective amount” will vary depending on the active agent or the compound, the condition being treated and its severity, the manner of administration, the duration of treatment, or the age of the subject to be treated, but can be determined routinely by one of ordinary skill in the art having regard to his own knowledge and to this disclosure.

The “sub-effective amount” or “sub-therapeutic amount” of a composition, extract mixture, component of the extract mixture, and/or active agent or ingredient or a therapy is an amount less than the effective amount for that composition, extract mixture, component of the extract mixture, and/or active agent or ingredient or therapy, but when combined with an effective or sub-therapeutic amount of another composition, extract mixture, component of the extract mixture, and/or active agent or ingredient can produce a desired result, due to, for example, synergy in the resulting efficacious effects, and/or reduced side effects.

The terms “synergistic effect” or “synergizing effect” is defined herein as the interaction of two or more combination compositions (e.g., an extract mixture) to produce a combined biological effect(s) greater than the sum of their separate effects (i.e., 1+1<2 or 1+1+1<3). The synergistic effect can be about or greater than about 10, 20, 30, 50, 75, 100, 120, 150, 200, 250, 350, or 500% or even more than the summed (additive) effect of each composition. The effect can be any of the measurable effects described herein.

As used herein, the term “carrier” refers to a composition that aids in maintaining one or more components (e.g., effective ingredients or active agents) of a composition in a soluble and homogeneous state in a form suitable for administration, which is nontoxic and which does not interact with other components in a deleterious manner. A carrier includes, but is not limited to, any materials known in the art including but not limited to any powder, liquid, gel, solubilizer, or binder. Some exemplary carriers include, but are not limited to, maltodextrin, gum arabic, starch, microcrystalline cellulose, hydroxypropyl methylcellulose, and mixtures thereof. In certain embodiments, the carrier is maltodextrin and/or the carrier that is used is taste and/or astringency neutral, i.e., it has no effect on palatability including astringency of the resulting product.

As used herein, the term “skin” refers to cell layers comprising the integument of a human or non-human individual, and its structural components such as hair, hair follicles, sebaceous glands, apocrine (sweat) glands, fingernails and toenails.

As used herein, the term “affected area of the skin” refers to a region of the skin that is to be treated with a composition including the active ingredients. For example, the affected area may be the site of a skin condition or disorder associated with skin oil production in an individual for which treatment or prevention is sought. In some cases, the affected area may encompass all skin on an individual. Alternatively, the affected area may be a site for which improvement of a cosmetic nature is sought, and can also include all skin on an individual or a specific area of the skin, such as face, back, or arms.

The term “skin condition or disorder associated with skin oil production” refers to any condition or disorder of the skin that results from either, increased or decreased skin oil production by the sebaceous glands of the skin. Examples of skin conditions or disorders associated with skin oil production include but are not limited to acne, sebaceous cysts, hyperplasia, blackheads, rough or uneven skin, enlarged and visible pores, eczema and the like.

As used herein, the term “systemic” or “systemically” refers to a mode of administration of a therapy via the blood stream or lymphatic system. Examples of a systemic treatment include, but are not limited to, oral gavage or ingestion, intravenous or subdermal pump infusion, and injection via intramuscular, intraperitoneal, hypodermic or subdermic injection.

As used herein, the term “topical” or “topically” refers to a mode of administration that is applied directly to an area of the skin, which may be the affected area of the skin. Examples of a topical treatment include, but are not limited to application of cream, lotion, gel, shampoo, conditioning lotion, spray, a pad, a bandage, a diaper, a moistened towelette, or transdermal patch; and local administration via intracutaneous injection or introduction of a lozenge or suppository.

The terms “treating” or “treatment” as used herein refer to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, (e.g., relieving pain, reducing inflammation, causing weight loss) without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The terms “administer,” “administered,” “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition may be preferred. Alternatively, topical administration may be preferable.

As used herein, the term “subject” or “individual” includes mammals to which a composition may be administered. Non-limiting examples of mammals include humans, non-human primates, rodents (including transgenic and non-transgenic mice) or the like. The methods described herein can be useful in human therapeutics, pre-clinical, nutraceutical, cosmetic and veterinary applications. In some embodiments, the subject is a mammal, and in some embodiments, the subject is human.

As used herein, “agent,” “active agent,” “biologically active agent,” or “active substance” refers to a biological, pharmaceutical, or chemical compound or other moiety obtained from a plant extract. Specifically, agent,” “active agent,” “biologically active agent,” or “active substance” may be a biological, pharmaceutical, or chemical compound or other moiety may be obtained from at least one of whole grape extract, honeybush extract and grape seed extract. Non-limiting examples include simple or complex organic or inorganic molecules, a peptide, a protein, a carbohydrate, a fatty acid or lipid like molecule.

The terms “modulator” refers to an active agent or a substance included as part of the composition, which modulates the activity or expression of one or more cellular proteins in a subject. A modulator may augment or suppress the activity and/or expression level or pattern of a molecule. A modulator can activate a component in a pathway by directly binding to the component. A modulator can also indirectly activate a component in a pathway by interacting with one or more associated components. The output of the pathway can be measured in terms of the expression or activity level of proteins. The expression level of a protein in a pathway can be reflected by levels of corresponding mRNA or related transcription factors as well as the level of the protein in a subcellular location. For instance, certain proteins are activated by translocating in or out of a specific subcellular component, including but not limited to nucleus, mitochondria, endosome, lysosome or other membranous structure of a cell. The output of the pathway can also be measured in terms of physiological effects, such as fat absorption and/or assimilation, weight loss, reduced skin oil production, etc.

An “activator” refers to a modulator that influences a pathway in a manner that increases the pathway output. Activation of a particular target may be direct (e.g. by interaction with the target) or indirect (e.g. by interaction with a protein upstream of the target in a signaling pathway including the target).

The terms “suppressor” or “inhibitor” refers to a modulator that influences a pathway in a manner that decreases pathway output. Inhibition of a particular target may be direct (e.g. by interaction with the target) or indirect (e.g. by interaction with a protein upstream of the target in a signaling pathway including the target).

### Compositions

A useful composition comprises at least physiologically acceptable quantity of an extract mixture that includes: (i) a whole grape extract that is able to at least partially inhibit DGAT-I activity, and (ii) at least one of: (a) at least physiologically acceptable quantity of a honeybush extract that is able to at least partially activate the PGCIα promoter activity and synergistically inhibit DGAT-I activity, and/or (b) at least physiologically acceptable quantity of a grape seed extract that is able to at least partially inhibit SREBPIc promoter activity and synergistically inhibit DGAT-I activity thereby having important ramifications in health and disease and a broad and important use in for weight management applications, as well, skin oil management applications.

Specifically, the described compositions can interfere with the absorption and assimilation of dietary fat by inhibiting the synthesis of fat that occurs within intestinal cells (not within the lumen of the gastrointestinal tract). The described compositions may also interfere with the synthesis of fat in adipose tissue within the body, a potential benefit observed that is not part of the effects of bile acid binding resins. In addition, the described compositions may interfere with the synthesis of fat in connection with the sebum production in the skin, and may have an effect of, for example, reducing excessive skin oil production in the instance of topical application.

The inhibition of absorption, assimilation and deposition of dietary fat may facilitate weight loss in individuals seeking to lose or maintain weight, and in whom high dietary fat intake is a challenge. For topical applications, the inhibition of fat production in sebum may improve or help to manage skin conditions associated with skin oil production (e.g., excessive skin oil production), such as skin acne.

In certain embodiments, a composition can include an effective amount of an extract mixture comprising a white grape extract and at least one of a honeybush extract and a grape seed extract.

In certain embodiments, the at least one of a honeybush extract and a grape seed extract are present in an amount with respect to the whole grape extract to synergistically inhibit diacylglycerol acyltransferase-I (DGAT-I).

In certain embodiments, the extract mixture comprises from about 0.5 weight percent (wt %) to about 90 wt % of the composition. In certain other embodiment, the extract mixture comprises from about 0.5 weight percent (wt %) to about 75 wt % of the composition. In certain other embodiment, the extract mixture comprises from about 0.5 weight percent (wt %) to about 50 wt % of the composition.

In certain embodiments, the composition includes at least about 5%, alternatively at least 10%, alternatively at least about 15%, alternatively at least about 20%, alternatively at least 25%, alternatively at least 30%, alternatively at least 40%, alternatively at least 45%, alternatively at least 50%, alternatively at least 55%, alternatively at least 60%, alternatively at least 65%, alternatively at least 70%, alternatively at least 75% of the extract mixture. In some embodiments, the extract mixture is present in an amount up to about 75%, alternatively, up to about 50%, alternatively up to about 55%, alternatively, up to about 40%, alternatively, up to about 30%, alternatively up to about 25% of the composition.

Sources of suitable whole grape extract have been obtained from Cyvex Nutrition, Irvine Calif., 92614. The whole grape extract may be prepared by extracting pulp, skin and seeds with water and ethanol to produce a yield of 8000:1 fresh whole grapes to whole grape extract ratio.

In one embodiment, the whole grape extract contains the following active ingredient(s): Proanthocyanidins, oligomeric Proanthocyanidins, polyphenols, gallic acid, trans-resveratrol, and stilbenoids. The whole grape extract may further include additional ingredients, such as procyanidins, catechins, catechin metabolites, anthocyanidines, and epicatechins.

The amount of polyphenols in the whole grape extract may vary from, e.g., 10% to 90%; more preferably from, e.g., 50% to 98%.

The amount of stilbenoids in the whole grape extract may vary from, e.g., 0.01% to 1%; more preferably from, e.g., 0.01% to 0.1%.

The amount of oligomeric Proanthocyanidins in the whole grape extract may vary from, e.g., 10% to 90%; more preferably from, e.g., 50% to 90%.

Preferably and advantageously, the whole grape extract comprises at least about 30%, alternatively at least about 35%, alternatively at least about 40%, alternatively at least about 45%, alternatively at least about 50%, alternatively at least about 55%, alternatively at least about 60%, alternatively at least about 65%, alternatively at least about 70%, alternatively at least about 75%, alternatively at least about 80%, alternatively at least about 90%, and alternatively about 100% of the extract mixture present in the present composition.

In certain embodiment, a composition comprises an effective amount of an extract mixture comprising a whole grape extract and a honeybush extract.

A suitable honeybush extract have been obtained by extraction methods from commercially sold Honeybush tea bags.

The honeybush extract includes the following active ingredients: flavanones and xanthones. The honeybush extract may further include additional ingredients, such as dihydrochalcones, flavones, and benzophenones.

The amount of magiferin in the honeybush extract may vary from, e.g., 0.1% to 10%; more preferably from, e.g., 0.5% to 5%.

The amount of hesperidin in the honeybush extract may vary from, e.g., 0.1% to 10%; more preferably from 0.5% to 5.0%.

The amount of luteolin in the honeybush extract may vary from 0.01% to 5.0%; more preferably from, e.g., 0.1% to 1%

Preferably, the whole grape extract comprises about 25% and the honeybush extract comprises about 75% of the extract mixture; more preferably, the whole grape extract comprises about 50% and the honeybush extract comprises about 50% of the extract mixture; more preferably, the whole grape extract comprises about 60% and the honeybush extract comprises about 40% of the extract mixture; most preferably, the whole grape extract comprises about 75% and the honeybush extract comprises about 25% of the extract mixture.

In certain embodiments, the ratio of the whole grape extract to the honeybush extract is 1:5; more preferably, the ratio is 1:4; more preferably, the ratio is 1:3; more preferably, the ratio is 1:2; more preferably, the ratio is 1:1; more preferably, the ratio is 2:1; more preferably, the ratio is 3:1; more preferably, the ratio is 4:1.

In certain other embodiments, a composition comprises an effective amount of an extract mixture comprising a whole grape extract and a grape seed extract.

Sources of suitable grape seed extract have been obtained from Polyphenolic, Madera, Ca 93637. The grape seed extract is prepared by extracting grape seeds with hot water to yield 30-50:1 grape seed to grape seed extract.

The grape seed extract includes the following ingredients: oligomeric anthocyanidines, proanthocyanidins, polyphenols, and gallic acid. The grape seed extract may further include additional ingredients, such as procyanidins, catechins, catechin metabolites and isomers, protocatechuic acid, epicatechins, epicatechins metabolites and isomers, and glucogallin.

The amount of polyphenols in the grape seed extract may vary from, e.g., 10% to 98%; more preferably from, e.g., 50% to 98%.

The amount of gallic acid in the grape seed extract may vary from, e.g., 1% to 50%; more preferably from, e.g., 5% to 20%.

The amount of catechins in the grape seed extract may vary from, e.g., 1% to 50%; more preferably from, e.g., 5% to 20%.

Preferably, the whole grape extract comprises about 25% and the grape seed extract comprises about 75% of the extract mixture; more preferably, the whole grape extract comprises about 50% and the grape seed extract comprises about 50% of the extract mixture; more preferably, the whole grape extract comprises about 60% and the grape seed extract comprises about 40% of the extract mixture; most preferably, the whole grape extract comprises about 75% and the grape seed extract comprises about 25% of the extract mixture.

In certain other embodiments, the ratio of the whole grape extract to the grape seed extract is 1:4; more preferably, the ratio is 1:3; more preferably, the ratio is 1:2; more preferably, the ratio is 1:1; more preferably, the ratio is 2:1; more preferably, the ratio is 3:1; more preferably, the ratio is 4:1.

In certain other embodiments, the extract mixture includes the whole grape extract and both, the honeybush extract and the grape seed extract, where the ratio of the whole grape extract to the honeybush extract and to the grape seed extract is 1:1:1; more preferably, the ratio is 1:1:2; more preferably, the ratio is 1:2:2; most preferably, the ratio is 1:1:2.5.

The compositions may be formulated for a systemic (e.g., oral) or topical administration or application. In some individuals it may be preferred to use a combination of systemic and topical administration. This can be due to the interest in controlling both topical and systemic triglyceride and fat synthesis.

To prepare the orally- or topically-administered compositions, an effective amount of the extract mixture including the whole grape extract and at least one of the honeybush extract or the grape seed extract is mixed with a pharmaceutically, nutraceutically or cosmetically acceptable vehicles, adjuvants, carriers and/or excipients (e.g., Starch, Maltodextrin, dextrin, Microcrystalline cellulose, Silicified microcrystalline cellulose, Cellulose). Pharmaceutically, nutraceutically or cosmetically acceptable vehicles, adjuvants, carriers and/or excipients are well known in the art, for example as described in the Handbook of Pharmaceutical Excipients, second edition, American Pharmaceutical Association, 1994 (incorporated herein by reference). Some specific examples are provided below.

In certain embodiments, the extracts may be incorporated into a solvent for ease of handling. For example, in a preferred embodiment, each of the whole grape extract, honeybush extract or grape seed extract is incorporated in a mixture of 1,3 butylene glycol and water, glycerin, propylene glycol, carbomer 980, cetyl and behenyl alcohol.

### Orally-Administered Compositions

The orally-administered compositions may be food or beverage compositions (e.g., health aid foods, nutrient supplements, or functional beverages), cosmetic compositions or pharmaceutical compositions.

In certain embodiments, the orally-administered compositions may include additional components, such as flavor, sweetener, colorant, and preservative components as well as supplement components.

a) Flavor, Sweetener, Colorant, and Preservative Components

Various additional components including natural and artificial flavors, natural and artificial colorants and/or food grade dyes can be included in the orally administered compositions. In addition, various preservatives, as would be understood by those of ordinary skill in the art can also be added.

A flavoring agent is adopted to enhance the taste or flavor of the composition and may be natural or synthetic. Preferable is a natural flavoring agent. A flavoring agent, if natural, may have the function of nutritional supplementation in addition to enhancing the flavor. Non-limiting examples of flavors include natural or artificial flavors and include chocolate; vanilla; caramel; coffee; fruit flavors including lemon, lime, orange, blackberry, raspberry, blueberry, peach, apricot, cherry, grape; creme, and mixtures thereof. Such flavors can be purchased, and/or prepared and added using known flavor technologies. The natural flavoring agent may be in the form of a liquid concentrate or a solid extract. A synthetic flavoring agent may be used, and is exemplified by esters, alcohols, aldehydes and terpenes.

The composition may contain an additive such as a sweetener. A sweetener is used to impart a sweet taste to the composition and may be natural or synthetic. Preferable is a natural sweetener. Examples of the natural sweetener include corn syrup, honey, sucrose, fructose, lactose, maltose and other sugars.

Non-limiting examples of suitable colorants include elderberry, caramel coloring made from caramelized sugar, Annatto, Chlorophyllin, Cochineal, Betanin, Turmeric, Saffron, Paprika, Lycopene, Pandan, and Butterfly pea.

Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, citric acid, ascorbic acid, malic acid, and mixtures thereof.

The compositions can include, individually or totally, at least about 0.001%, by weight of the composition, of flavor, colorant, and/or preservative components, and mixtures thereof. Alternatively, the compositions can include, individually or totally, from about 0.001% to about 10%, alternatively from about 0.001% to about 5%, alternatively from about 0.01% to about 4%, alternatively from about 0.1% to about 3% by weight of the composition, of each or the flavor, colorant components, and/or preservative components and mixtures thereof.

b) Supplement Components

While the present composition is primarily intended to be a composition of the extract mixture, it is contemplated that embodiments of the invention can include supplements such as, but not limited to, vitamins, minerals, herbs, botanicals, plant derived supplements, animal derived supplements, therapeutic compounds, and mixtures thereof.

Non-limiting examples of such other components include: calcium, potassium, B vitamins, vitamins A, C, D, E, and K, folic acid, other vitamins and minerals commonly known in the art and used for supplementing the diet (e.g., magnesium, chrome, cobalt, copper, fluorides, germanium, iodine, iron, lithium, magnesium, manganese, molybdenum, phosphorus, calcium, selenium, silicone, sodium, sulfur, vanadium, and zinc); extracts and active phytochemicals including ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine; glucosamine, chondroitin; melatonin, seratonin; and mixtures thereof.

The compositions can include at least about 0.001%, by weight of the composition, of a supplement component. Alternatively, the composition can include from about 0.001% to about 25%, alternatively from about 0.01% to about 10%, and alternatively from about 0.1% to about 5%, by weight of the composition, of a supplement component.

The agents such as preservatives, emulsifiers, etc. are used in as minimal an amount as possible to achieve the purpose of their addition. Numerically, their amount ranges from approximately 0.0005% by weight to 0.5% by weight based on the total weight of the composition.

In certain embodiments, the pharmaceutical composition comprises a pharmaceutically acceptable vehicle or excipient in addition to the active ingredient and may be formulated into oral dosage forms (tablets, suspensions, granules, emulsions, capsules, syrup, etc.), parenteral dosage forms (sterile injections, aqueous or oily suspensions, etc. Examples of the pharmaceutically acceptable vehicle were provided above. The vehicles may be used, individually or in combination, according to the formulation of the pharmaceutical composition.

A suitable excipient may also be employed in the pharmaceutical composition. For example, an excipient suitable for formulating the pharmaceutical composition into an aqueous suspension may be a suspending agent or dispersant such as sodium carboxymethyl cellulose, methyl cellulose, hydropropylmethylcellulose, sodium alginate, or polyvinylpyrrolidone. When the pharmaceutical composition is formulated into an injection, Ringer's solution, or isotonic sodium chloride may be used as an excipient.

To administer the pharmaceutical composition, an oral route or a parenteral route such as a topical route may be taken.

The food or pharmaceutical orally-administered compositions can be formed into any suitable, ingestible form. Non-limiting examples of the form of the compositions include: soft chew, hard chew, chewable tablet, nutritional bar, lozenge, powder, granules, clusters, soft gel, semi-solid taffy-like chew, chewing gum, swallowable tablet, swallowable capsule, swallowable caplet, individual unit doses, user-dosable forms, and mixtures thereof. For example, a unit dose can be a single soft chew, or a partitionable form such as a bar which the user cuts or breaks to provide unit dosages.

“Soft chew” is intended to mean a product which is solid at room temperature and which are soft to chew and which is functionally chewy because the product has some plastic texture during the process of mastication in the mouth.

In certain embodiments, the composition is free of unbound or added water. The term “unbound water” refers to water that is not present as part of an ingredient used in the composition. In contrast, “bound water” refers to that water that is present as part of the ingredient, e.g., water that might be present as part of a fruit juice concentrate.

In certain embodiments, the orally administered composition may be in a form of syrup, food (such as food bars, biscuits, snack foods and other standard food forms well known in the art) or a drink or a beverage. Drinks can contain flavoring, buffers and the like, as described above.

3. Dosages

The daily dose of a food or pharmaceutical composition may be, e.g., 0.25 to 2.0 g/day.

A single dose or multiple doses per day may be administered.

The dose of the pharmaceutical composition may vary depending on various factors including the route of administration, the patient's age, gender and weight, and the severity of illness and thus must be in no way understood as limiting the scope.

Similarly, the dose (such as an amount) of the food or beverage composition may also vary depending on various factors including the patient's age, gender and weight, and the severity of illness and thus must be in no way understood as limiting the scope.

Preferably, the food or beverage or pharmaceutical composition is administered for consumption immediately prior to ingestion of dietary fat. Alternatively, the food or beverage or pharmaceutical composition is administered for consumption at least 15 minutes prior to ingestion of dietary fat; alternatively, at least 30 minutes prior to ingestion of dietary fat; alternatively, at least 45 minutes prior to ingestion of dietary fat; alternatively, at least 1 hour prior to ingestion of dietary fat; alternatively, at least 1.5 hours prior to ingestion of dietary fat; alternatively, at least 2 hours or more prior to ingestion of dietary fat.

In an alternative embodiment, the food or beverage or pharmaceutical composition is administered for consumption immediately following the ingestion of dietary fat. Alternatively, the food or beverage or pharmaceutical composition is administered for consumption less than about 2 hours after ingestion of dietary fat; alternatively, less than about 1.5 hours after the ingestion of dietary fat; alternatively, less than about 1 hour after the ingestion of dietary fat; alternatively, less than about 45 minutes after the ingestion of dietary fat; alternatively, less than about 30 minutes after the ingestion of dietary fat; alternatively, less than about 15 minutes after the ingestion of dietary fat; alternatively, less than about 5 minutes after the ingestion of dietary fat.

In certain embodiments, the food or beverage or pharmaceutical composition is administered for consumption concurrently with the ingestion of dietary fat.

The food or beverage or pharmaceutical composition is administered for consumption for a suitable time period to achieve the desired effect, such as reduction in body weight or reduction in unwanted skin oil production.

For example, the food or beverage or pharmaceutical composition may be administered for consumption for 1 day, 2 days, 5 days, a week, two weeks, a month or more. The food or beverage or pharmaceutical composition may also be administered for consumption for an indefinite period of time.

### Topically-Administered Compositions

In certain embodiments, the compositions include an extract mixture that includes a whole grape extract in combination with either honeybush extract or grape seed extract, where the composition is for topical administration.

The topical administration may be suitable for treating, preventing or managing skin conditions or disorders associated with skin oil production in a subject.

For example, in accordance with one aspect, the rate of skin oil production may be inhibited by topical application to the skin of the compositions to the affected area, such as face, back or arms. The topical administration of the composition may be to affected areas where skin oil production is excessive and where less skin oil production is desired.

Generally, the topical application is on at least once daily basis; alternatively, the topical application is on at least twice daily basis; more preferably, three times a day or more.

Generally, the composition may be applied for any suitable period of time. For example, the composition may be applied for a few minutes to several minutes or hours. The application may be continuous or intermittent.

Within a few days, a user may notice improvement in skin oil production. The user may also notice additional improvements as far as skin texture and smoothness.

The compositions for topical applications may be formulated as a solution, gel, lotion, cream, ointment, oil-in-water emulsion, water-in-oil emulsion, or other pharmaceutically, nutraceutically or cosmetically acceptable form for topical application.

The compositions may also contain various known and conventional cosmetic and pharmaceutical ingredients so long as they do not detrimentally affect the desired skin effect. For example, the cosmetically acceptable vehicle may acts as a dilutant, dispersant or carrier for other materials present in the composition, so as to facilitate their distribution when the composition is applied to the skin.

In certain embodiments, a topical composition may also include other cosmetic and pharmaceutical actives and excipients.

Vehicles other than water can include liquid or solid emollients, solvents, humectants, thickeners and powders. As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials.

Vehicles may also include propellants such as propane, isobutane, dimethyl ether, carbon dioxide, nitrous oxide; and solvents such as ethyl alcohol, isopropanol, acetone, ethylene glycol monomethyl ether, diethylene glycol monobutyl ether, diethylene glycol monoethyl ether, or powders such as chalk, talc, fullers earth, kaolin, starch, gums, collodial silica, sodium polyacrylate, tetra alkyl and/or trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, ethylene glycol monostearate.

Examples of suitable cosmetic and pharmaceutical agents for use with the topically administered composition include, but are not limited to, antifungals, vitamins, anti-inflammatory agents, antimicrobials, analgesics, nitric oxide synthase inhibitors, insect repellents, self-tanning agents, surfactants, moisturizers, stabilizers, preservatives, antiseptics, thickeners, lubricants, humectants, chelating agents, skin penetration enhancers, emollients, fragrances and colorants.

The composition can optionally comprise sunscreens such as inorganic and organic sunscreens to provide protection from the harmful effects of excessive exposure to sunlight during use of the composition.

Examples of suitable organic sunscreens, when required, include those set out in the current OTC Sunscreen Monograph, which is incorporated herein by reference. The composition of the invention can accordingly comprise from 0.1 to 10%, preferably from 1 to 5% by weight of an organic sunscreen material.

The composition optionally can also comprise inorganic sunscreens such as titanium dioxide, zinc oxide, having an average particle size of from 1 to 300 nm, iron oxide, having an average particle size of from 1 to 300 nm, silica, such as fumed silica, having an average particle size of from 1 to 100 nm. It should be noted that silica, when used as an ingredient in the emulsion according to the invention can provide protection from infrared radiation.

Ultrafine titanium dioxide in either of two forms, namely water-dispersible titanium dioxide and oil-dispersible titanium dioxide may be used. Water-dispersible titanium dioxide is ultrafine titanium dioxide, the particles of which are uncoated or which are coated with a material to impart a hydrophilic surface property to the particles. Examples of such materials include aluminum oxide and aluminum silicate. Oil-dispersible titanium dioxide is ultrafine titanium dioxide, the particles of which exhibit a hydrophobic surface property, and which, for this purpose, can be coated with metal soaps such as aluminum stearate, aluminum laurate or zinc stearate, or with organosilicone compounds.

The term “ultrafine titanium dioxide” refers to particles of titanium dioxide having an average particle size of less than 100 nm, preferably from 10 to 40 nm and most preferably from 15 to 25 nm. The total amount of titanium dioxide that can, optionally, be incorporated in the composition according to the invention is from 1 to 25%, preferably from 2 to 10% and ideally from 3 to 7% by weight of the composition.

A product for topical application can comprise at least 0.01%, and up to 10%, by weight extract mixture. Selected concentration ranges include from about 0.01% to about 3%, from about 0.1% to about 1%, from about 0.1% to about 3%, from about 0.1% to about 5%, from about 0.3% to about 1%, from about 0.3% to about 3%, from about 0.3% to about 5%, from about 0.5% to about 1%, from about 0.5% to about 3%, and from about 0.5% to about 5%. Typically, 0.01% to 1% is an effective concentration range that can be applied at a variety of intervals. In some cases, it is preferred to apply the product in a concentration of up to 5% to treat some pathological conditions or diseases. There are also instances in which a concentration of up to 10% may be required, due to the severity of a condition or disease.

### Uses and Products

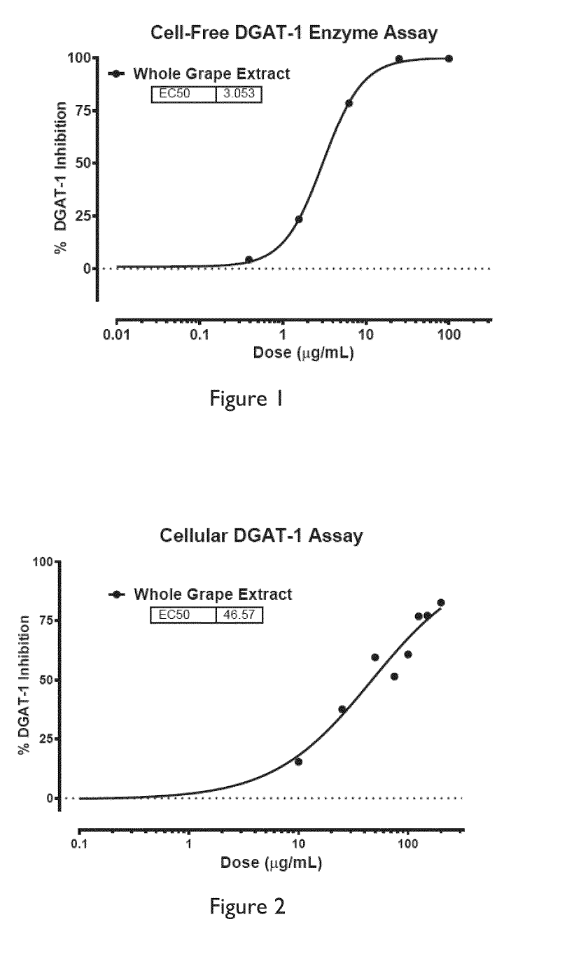

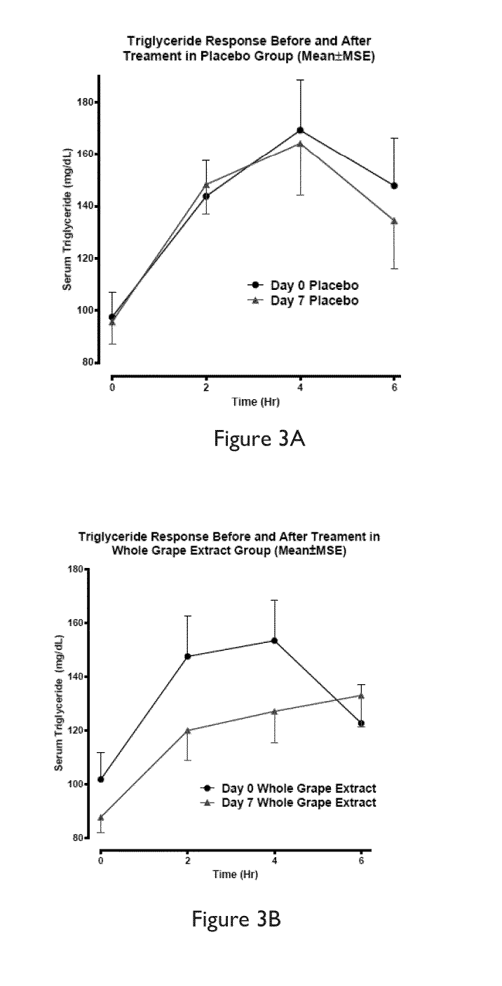

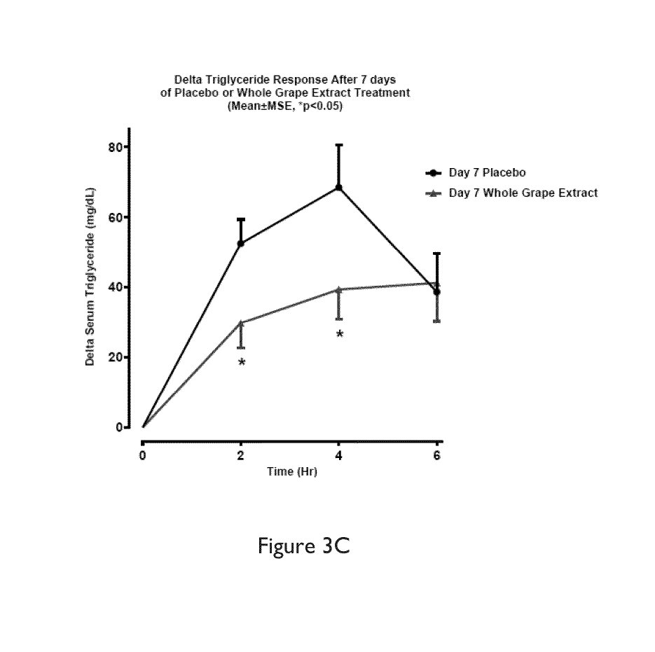

Whole grape extracts were identified as potent inhibitors of an enzyme that catalyzes the terminal step in mammalian triglyceride (fat) synthesis, diacylglycerol acyltransferase-I or DGAT-I. Specifically, whole grape extracts were identified to be potent inhibitors of DGAT-I activity in both, the cell-free and cellular DGAT-I assays (see FIGS. 1 and 2). When the whole grape extract was tested in a proof-of-mechanism human clinical trial, it resulted in a characteristic response in vivo DGAT-I inhibition, including: (1) decreased fasting triglycerides, (2) reduced response to oral fat challenge, and (3) delayed time to maximum triglyceride concentration (see FIG. 3).

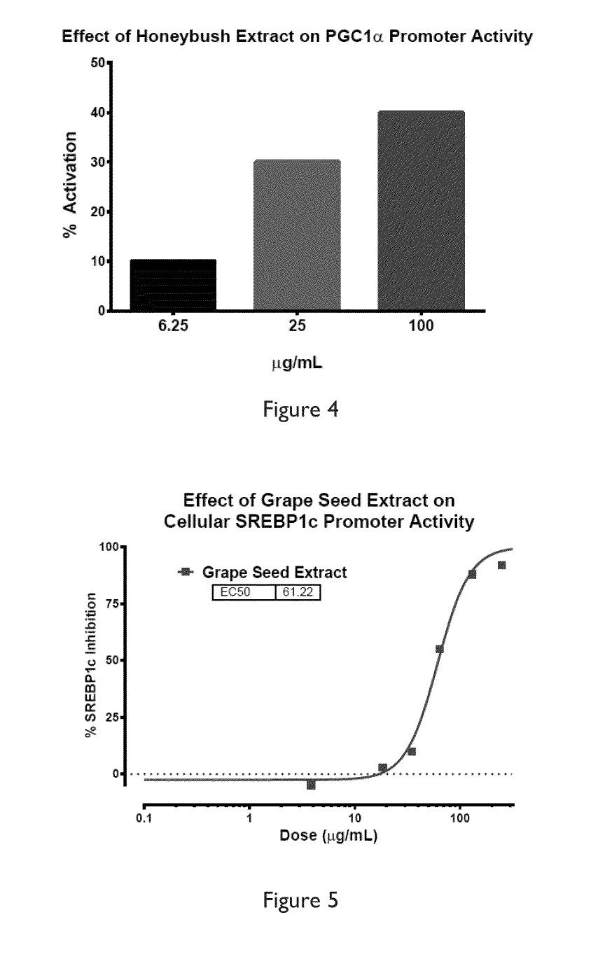

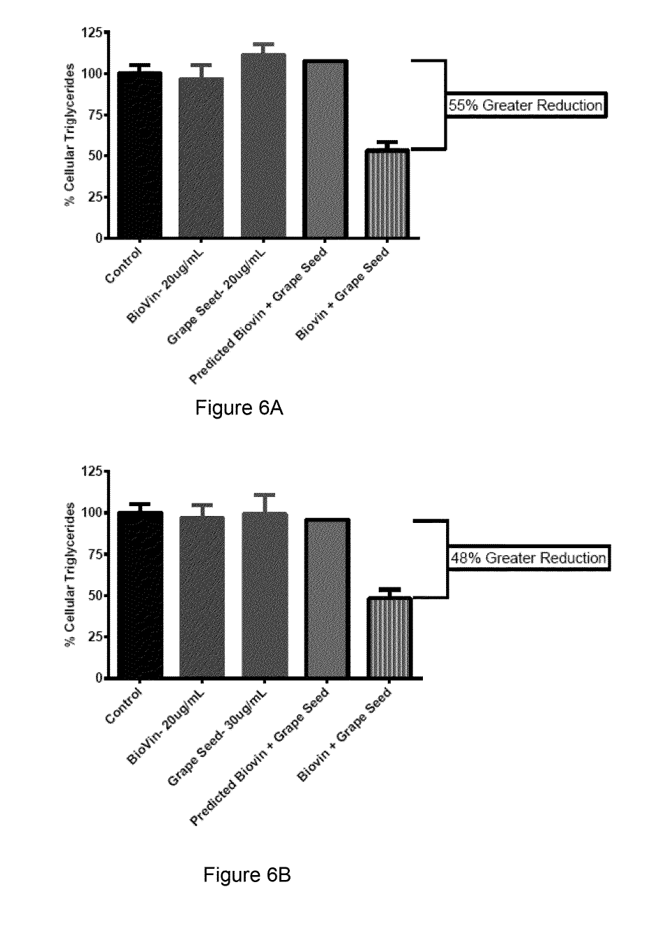

In addition, two complementary mechanisms that could synergize with DGAT-I to inhibit lipid formation and subsequent adipose tissue formation were identified. The two targets were identified as peroxisome proliferator-activated receptor gamma coactivator I-alpha (PGCIα) and sterol regulatory element-binding protein Ic (SREBPIc). Botanical extracts of honeybush and grape seed were identified as an activator of the PGCIα promoter activity (FIG. 4) and an inhibitor of SREBPIc promoter activity, respectively (see FIGS. 4 and 5).

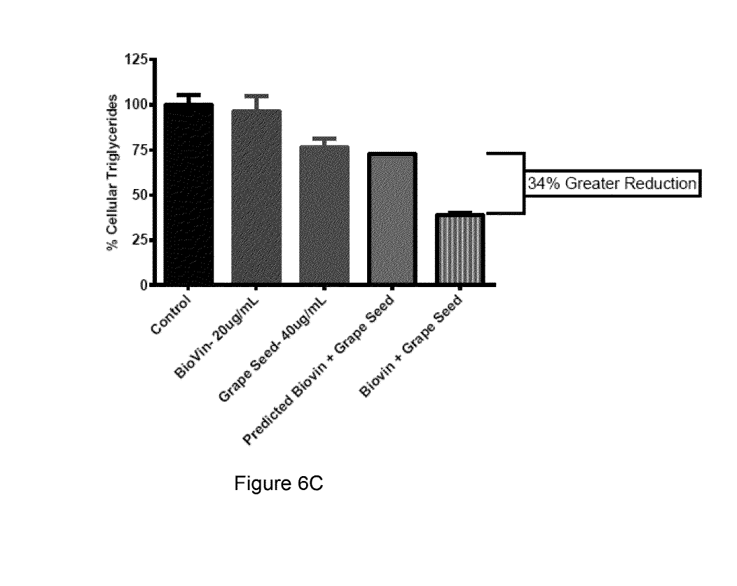

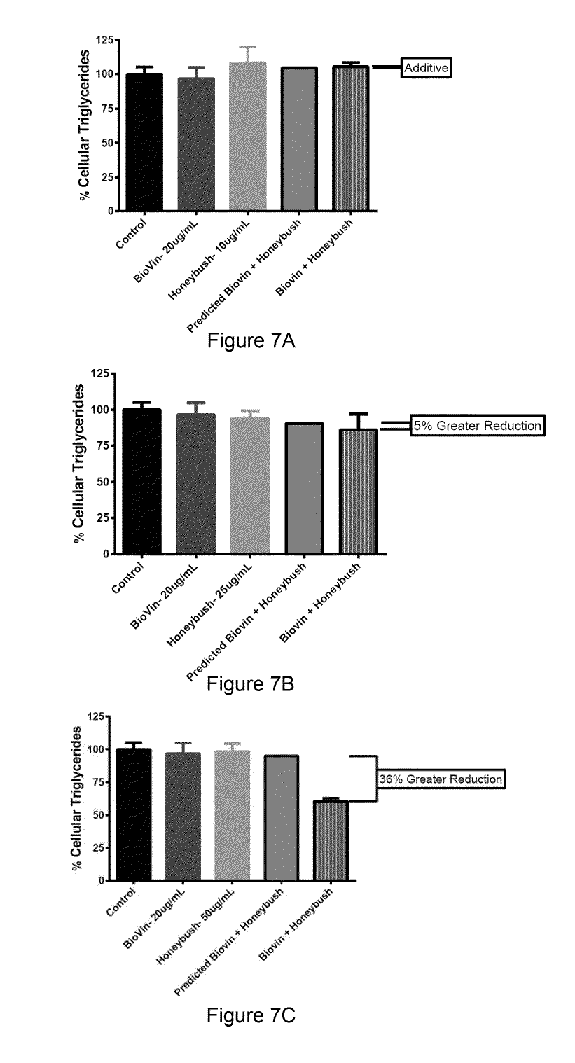

In a series of synergy experiments to examine the potential synergistic effects of combining whole grape extract with either the honeybush extract or the grape seed extract, both extracts provided synergy to the whole grape extract on cellular DGAT-I activity (see FIGS. 6 and 7).

In view of this synergistic effect on the DGAT-I enzyme activity and further inhibitory effect on SREBPIc and activation of PGCIα, the compositions may be used in weight management applications, and for topical products, control of skin oil applications.

Certain embodiments include the use of the present composition to modulate the DGAT-I enzyme, for example, to inhibit the DGAT-I enzyme.

Certain embodiments relate to the use of the present composition to modulate dietary fat absorption and assimilation in a subject.

Other embodiments relate to the use of the present composition to suppress the synthesis of fat in adipose tissue.

Other embodiments relate to the use of the present composition to modulate production of skin oil and to the use of the present composition to inhibit production of skin oil.

Certain embodiments relate to a weight management product that includes a composition that includes an effective amount of an extract mixture, where the extract mixture includes the whole grape extract and at least one of the honeybush extract and grape seed extract. The product may be a food or a drink that includes the composition as an active substance. Alternatively, the product may be a pharmaceutical that includes the composition as the active substance. In yet other embodiments, the product may be formulated for topical administration. The product may also be formulated for cosmetic use. Specific examples of formulations, modes of administration, types and dosages were described in detail above.

Certain other embodiments relate to methods for treating, preventing, or managing body weight gain in a subject. The method includes administering to the subject a composition that includes an effective amount of an extract mixture comprising a whole grape extract and at least one of a honeybush extract and a grape seed extract to effectively treat, prevent, or manage weight gain in the subject.

A further embodiment relates to a method of reducing fat absorption, transport, deposition, production and secretion in a subject. The method includes administering to the subject a composition that includes an effective amount of an extract mixture that includes a whole grape extract and at least one of a honeybush extract and a grape seed extract to effectively reduce fat absorption, transport, deposition, production and secretion in the subject.

A further embodiment relates to a method of facilitating body weight lost in a subject. The method includes administering to the subject a composition that includes an effective amount of an extract mixture that includes a whole grape extract and at least one of a honeybush extract and a grape seed extract to effectively facilitate weight loss in the subject.

Certain other embodiments relate to a method of maintaining body weight of a subject. The method includes administering to the subject a composition that includes an effective amount of an extract mixture that includes a whole grape extract and at least one of a honeybush extract and a grape seed extract to effectively maintain body weight of the subject.

Another embodiment relates to a method for treating, preventing or managing skin conditions or disorders associated with skin oil production in a subject. The method includes administering to the subject a composition that includes an effective amount of an extract mixture that includes a whole grape extract and at least one of a honeybush extract and a grape seed extract to effectively treat, prevent or manage skin conditions or disorders associated with skin oil production in the subject. The skin condition or disorder associated with skin oil production may be one resulting from excessive skin oil production, such as clogged hair follicles and pores, blackheads, and resulting acne that usually form on the face, but can also appear on the back, chest, neck, arms, and shoulders.

Another embodiment relates to a method of synergistically inhibiting DGAT-I in a subject. The method includes administering to the subject a composition comprising an effective amount of an extract mixture that includes a whole grape extract and at least one of a honeybush extract and a grape seed extract to synergistically inhibit DGAT-I in the subject.

### EXAMPLES

A better understanding may be obtained through the following examples which are set forth to illustrate, but are not to be construed as limiting.

Methods

The whole grape extract is prepared by extracting pulp, skin and seeds with water & ethanol to produce a yield of 8000:1 fresh whole grapes to whole grape extract ratio.

Dried Honeybush leaves and stems, Numi Organic Tea bags, were extracted in 83% ethanol/water at a 1:10 material to alcohol ratio at 140° F. for 2 hours with constant mixing. The slurry was then cooled and passed through a 325 mesh screen and the resulting cake was hand pressed. All liquid extracts collected were combined and the remaining alcohol was removed by evaporation. Water was added to approximately double the amount of liquid concentrate.

An extraction of the grape seed was performed by extracting grape seed with hot water at 30-50:1 grape seed to solvent ratio.

### Example 1

### Identification of Potent Inhibitors of DGAT-1

More than 200 botanical extracts were evaluated in a cell-free DGAT-I enzyme assay for properties to inhibit the DGAT-I enzyme activity. The botanical extracts were also evaluated in a cellular DGAT-I assay model system.

DGATI Enzyme Assay Protocol:

Microsomes from Human intestinal cells were used as the source of DGATI enzyme.

Dioleoyl Glycerol (DG) and Palmitoleoyl-Coenzyme A (PCoA) were the substrates for DGATI. Substrates (DG (300 uM)+PCoA (75 uM)) were mixed with botanical extracts, vehicle or positive control (A922500) in a 1.5 mL tube. Microsomes (22.5 ug/mL) were added, vortexed and incubated in a 37° C. water bath for 1 hour. After 1 hour, Dioeloyl-Palmitolyl glycerol (TG product formed from DGATI enzyme reaction) and Trioeloyl glycerol (internal standard) were extracted with Isopropyl Alcohol/Methylene Chloride/Formic Acid. Extracted TGs were filtered and then separated and identified by Liquid Chromatography Mass Spectrometer (LCMS) using Acetonitrile/Isopropyl Alcohol/Methylene Chloride/Formic Acid/Ammonium Hydroxide Mobile Phase. Dioeloyl-Palmitolyl glycerols were quantitated relative to internal standard.

Cellular DGATI Assay Protocol:

293H cells were plated at confluence in 12 well culture plates and allowed to adhere overnight. The next morning, media was replaced with serum free medium containing botanical extracts alone and in combination for synergy experiments or controls (Vehicle, A922500 (positive control)) and allowed to incubate for 30 minutes (pretreatment). After pretreatment, triglyceride (TG) synthesis was induced with 0.3 mM oleic acid/BSA containing [14C]-glycerol (1 uCi/ml) and allowed to incubate for 5 hours at 37° C., 5% CO2. Control cells pretreated with vehicle had serum-free medium+10% fatty acid free BSA and [14C]-glycerol added to control for oleic acid induction. After 5 hours, medium was removed and cells washed, dislodged and collected in a small volume of PBS.

Samples were prepped for thin Layer Chromatography (TLC) by extraction with chloroform/methanol and the organic layer washed before an aliquot of the final organic phase is dried to completion. Lipids are solubilized with a small amount of chloroform containing TG, 1,2- and 1,3-DAG, and MAG standards and separated by TLC using toluene/chloroform/methanol mobile phase. Lipid species were identified by iodine vapor and comparison to standards. Radioactivity incorporated into TG was determined by scraping and counting by MicroBeta TriLux.

Whole Grape Extracts were identified as potent inhibitors of DGAT-I activity in both the cell-free and cellular DGAT-I assays (see FIGS. 1 & 2).

Specifically, FIG. 1 shows the dose response and EC50 value (3.053 μg/mL) of whole grape extract on DGAT-I enzyme inhibition in a cell-free assay.

FIG. 2 shows the dose response and EC50 (46.574 mL) value of the whole grape extract on cellular DGAT-I inhibition.

### Example 2

### Proof of Mechanism Human Clinical Trial

Human Proof of Mechanism (POM) Clinical Trial Design:

The clinical trial was a 2-arm parallel, double-blind study of 37 healthy overweight/obese (BMI of 25-35 kg/m²) men and women that were randomized to receive 2 grams daily of whole grape extract or placebo for a period of 7 days. Prior to and after the 7 day intervention with whole grape extract or placebo, study subjects consumed a meal challenge containing a standardized triglyceride load (84% Fat, 12% Carbohydrate, 4% protein). Blood samples were taken before and immediately following the meal challenge across a six hour interval and were evaluated for serum triglycerides levels, which is a validated measure of DGATI activity (Pharmacological inhibition to examine the role of DGATI in dietary lipid absorption in rodents and humans. Benjamin S. Maciejewski et al., American Journal of Physiology—Gastrointestinal and Liver Physiology 2013 Vol. 304: G958-G969 DOI: 10.1152/ajpgi.00384.2012).

When whole grape extract was tested in a Proof of Mechanism Human clinical trial, it resulted in a characteristic response of in vivo DGAT-I inhibition, including: (1) decreased fasting triglycerides, (2) reduced response to oral fat challenge and (3) delayed time to maximum triglyceride concentration (see FIGS. 3A-C).

Specifically, as shown in FIG. 3A, seven days of placebo treatment did not affect serum triglyceride response to an oral fat challenge, which is a validated method to examine DGAT-I activity in vivo.

As shown in FIG. 3B, treatment with 2 grams of whole grape extract showed a significant percent reduction in triglyceride response at the conclusion of the treatment period (7 days) as compared to the triglyceride response before the initiation of the study.

As shown in FIG. 3C, similarly, a 7 day treatment with whole grape extract resulted in a significant delta reduction in triglyceride response as compared to the placebo.

TABLE I
Means and Std Deviations, Delta Triglyceride (TG).
After 7 days of Treatment

| | | Mean | | | | | |
|---|---|---|---|---|---|---|---|
| Time after | | Delta | | Std | | | |
| oral fat | | TG, | Std | Err | Lower | Upper | p-value |
| load | Group | mg/mL | Dev | Mean | 95% | 95% | (t-test) |
| 2 | Whole | 29.80 | 27.28 | 7.04 | 14.69 | 44.91 | 0.0139* |
| | Grape | | | | | | |
| | Extract | | | | | | |
| | Placebo | 52.53 | 28.27 | 6.86 | 37.99 | 67.07 | |
| 4 | Whole | 39.40 | 33.02 | 8.52 | 21.12 | 57.68 | 0.0299* |
| | Grape | | | | | | |
| | Extract | | | | | | |
| | Placebo | 68.47 | 49.99 | 12.12 | 42.77 | 94.17 | |
| 6 | Whole | 41.27 | 42.45 | 10.96 | 17.76 | 64.78 | 0.5650 |
| | Grape | | | | | | |
| | Extract | | | | | | |
| | Placebo | 38.71 | 45.22 | 10.97 | 15.46 | 61.96 | |

### Example 3

### Effects of the Honeybush Extract on the PGCIα Promoter Activity and Grape Seed Extract on SREBPIc Promoter Activity

Next, two complementary mechanisms that could synergize with DGAT-I to inhibit lipid formation and subsequent adipose tissue formation were identified.

The two targets were peroxisome proliferator-activated receptor gamma coactivator I-alpha (PGCIα) and sterol regulatory element-binding protein Ic (SREBPIc).

A. PGCIα Promoter Activity

CHO cell line was stably transfected with a luciferase reporter vector (pGL4.27) under the control of the full length human PGCIα promoter. Stable cells were grown and treated with extracts in a 96 well plate for 18 hr. After 18 hrs of incubation, the medium from the cell were removed and the cells washed once with 200 ul of PBS (Phosphate buffered saline). Then 20 μl of passive lysis buffer was added per well and incubated at room temperature to lyse the cells. Subsequent to this, 100 μl of luciferase substrate buffer was added and the luminescence was read in a plate reader.

A botanical extract (Honeybush) that activated the PGCIα promoter activity was identified (FIG. 4).

Specifically, FIG. 4 shows a dose response activation of the cellular PGCIα promoter activity by honeybush extract.

B. SREBPIc Promoter Activity

HEPG2 cell line was stably transfected with a luciferase reporter vector (pGL4.27) under the control of the full length human SREBPIc promoter. Stable cells were grown and treated with extracts in a 96 well plate for 18 hr. After 18 hrs of incubation, the medium from the cell were removed and the cells washed once with 200 ul of PBS (Phosphate buffered saline). Then 20 μl of passive lysis buffer was added per well and incubated at room temperature to lyse the cells. Subsequent to this, 100 μl of luciferase substrate buffer was added and the luminescence was read in a plate reader.

A botanical extract (Grape Seed) that inhibited SREBPIc promoter activity was identified (FIG. 5).

Specifically, FIG. 5 shows a dose response inhibition of the SREBPIc promoter activity by grape seed extract.

### Example 4

### Synergy with the Grape Seed Extract

Potential synergistic effects of combining the whole grape extract with the grape seed extract were examined.

20 μg/mL dose of grape extract, various doses of the grape seed extract (20 μg/mL, 30 μg/mL and 40 μg/mL) and a low dose the Whole grape extract (20 μg/mL) in combination with various doses of grape seed were tested to determine the effect on the cellular triglyceride formation (FIGS. 6A-C; data is expressed as percent of untreated control cells).

As shown in FIGS. 6A-C, significant synergistic responses were apparent at all doses of grape seed extract tested in combination with 20 μg/mL dose of the whole grape extract.

As shown in FIG. 6A, the 20 μg/mL dose of the Whole grape extract in combination with the lowest dose of the grape seed extract (20 μg/mL) resulted in a significant reduction in cellular triglyceride formation as compared to the control, the Whole grape extract alone, and the grape seed extract alone. When the result was compared to the “predicted” additive effect (i.e., effect of the Whole grape extract alone in combination with effect of the grape seed extract alone), a maximal synergy was seen at that dose, 55% reduction (FIG. 6A).

As shown in FIG. 6B, the whole grape extract in combination with the grape seed extract at 30 μg/mL resulted in 48% reduction in cellular triglyceride formation as compared to the predicted result for the combination.

As shown in FIG. 6C, the whole grape extract in combination with the grape seed extract at 40 μg/mL resulted in 34% reduction in cellular triglyceride formation as compared to the predicted result for the combination.

### Example 5

### Synergy with the Honeybush Extract

Potential synergistic effects of combining the whole grape extract with the honeybush extract were examined.

20 μg/mL dose of the Whole grape extract, various doses of honeybush extract (10 μg/mL, 25 μg/mL and 50 μg/mL) and a low dose Whole grape extract (20 μg/mL) in combination with various doses of the honeybush extract were tested to determine the effect on the cellular triglyceride formation (FIGS. 7A-C; data is expressed as percent of untreated control cells).

As shown in FIGS. 7A-C, a significant synergistic response was apparent a high dose of the honeybush extract tested in combination with the whole grape extract at the 20 μg/mL dose.

As shown in FIG. 7A, the Whole grape extract in combination with the honeybush extract at 10 μg/mL resulted in no observable effect on cellular triglyceride formation as compared to the control, the whole grape extract alone, the honeybush extract alone, and the predicted result.

As shown in FIG. 7B, the whole grape extract in combination with the honeybush extract at 25 μg/mL resulted in about 5% reduction in cellular triglycerides as compared to the predicted result.

As shown in FIG. 7C, the 20 μg/mL dose of the whole grape extract in combination with the highest dose of the honeybush extract (50 μg/mL) resulted in a significant reduction in cellular triglyceride formation as compared to the control, the Whole grape extract alone, and the honeybush extract alone. When the result was compared to the “predicted” additive effect (i.e., effect of Whole grape extract alone in combination with the effect of the honeybush extract alone at 50 μg/mL), a maximal synergy was seen at that dose, about 36% reduction (FIG. 7C).

The described experiments confirm the synergistic effect of an extract mixture that includes the whole grape extract with either honeybush extract or the grape seed extract on cellular formation of the triglycerides via inhibition of the DGAT-I enzyme.

It is therefore intended that the foregoing detailed description be regarded as illustrative rather than limiting, and that it be understood that it is the following claims, including all equivalents, that are intended to define the spirit and scope of this invention.

1. A composition comprising an effective amount of an extract mixture comprising a whole grape extract and at least one of a honeybush extract and a grape seed extract.

2. The composition of claim 1, wherein the at least one of a honeybush extract and a grape seed extract are present in an amount with respect to the whole grape extract to synergistically inhibit diacylglycerol acyltransferase-I (DGAT-I).

3. The composition of claim 1, wherein the honeybush extract is present in an amount to activate PGCIα promoter activity.

4. The composition of claim 1, wherein the grape seed extract is present in an amount to inhibit SREBPIc promoter activity.

5. The composition of claim 1, further comprising a pharmaceutically, nutraceutically or cosmetically acceptable carrier, diluent, or excipient, wherein the extract mixture comprises from about 0.5 weight percent (wt %) to about 90 wt % of the composition.

6. The composition of claim 1, further comprising a pharmaceutically, nutraceutically or cosmetically acceptable carrier, diluent, or excipient, wherein the extract mixture comprises from about 0.5 weight percent (wt %) to about 75 wt % of active ingredients of the composition.

7. The composition of claim 1, further comprising a pharmaceutically, nutraceutically or cosmetically acceptable carrier, diluent, or excipient, wherein the extract mixture comprises from about 0.5 weight percent (wt %) to about 50 wt % of active ingredients of the composition.

8. The composition of claim 1, wherein the composition is for oral administration.

9. The composition of claim 8, wherein the composition is formulated as a tablet, capsule, powder, or granule.

10. The composition of claim 1, wherein the composition is for topical administration.

11. The composition of claim 10, wherein the composition is formulated as a cream, gel, lotion, spray solution, pad, bandage, and a transdermal patch.

12. The composition of claim 1, wherein the composition modulates dietary fat absorption and assimilation in a subject.

13. The composition of claim 1, wherein the composition suppresses the synthesis of fat in adipose tissue.

14. The composition of claim 1, wherein the composition inhibits diacylglycerol acyltransferase-I enzyme.

15. The composition of claim 1, wherein the composition modulates production of skin oil.

16. The composition of claim 1, wherein the ratio of the whole grape extract to the honeybush extract or a grape seed extract is 1:1.

17. The composition of claim 1, wherein the ratio of the whole grape extract to the honeybush extract or a grape seed extract is 1:2.

18. A food or a drink comprising the composition of claim 1.

19. A weight management product comprising a composition comprising an effective amount of an extract mixture comprising a whole grape extract and at least one of a honeybush extract and a grape seed extract.

20. The weight management product of claim 19, wherein the product is formulated for oral administration.

21. The weight management product of claim 19, wherein the product is a food or a drink comprising the composition as an active substance.

22. The weight management product of claim 19, wherein the product is a pharmaceutical comprising the composition as an active substance.

23. The weight management product of claim 19, wherein the product is formulated for topical administration.

24. The weight management product of claim 19, wherein the product is formulated for cosmetic use.

25. The weight management product of claim 19, further comprising a pharmaceutically, nutraceutically or cosmetically acceptable carrier, diluent, or excipient, wherein the extract mixture comprises from about 0.5 weight percent (wt %) to about 90 wt % of the composition.

26. The weight management product of claim 19, further comprising a pharmaceutically, nutraceutically or cosmetically acceptable carrier, diluent, or excipient, wherein the extract mixture comprises from about 0.5 weight percent (wt %) to about 75 wt % of the composition.

27. The weight management product of claim 19, further comprising a pharmaceutically, nutraceutically or cosmetically acceptable carrier, diluent, or excipient, wherein the extract mixture comprises from about 0.5 weight percent (wt %) to about 50 wt % of the composition.

28. A method for treating, preventing, or managing body weight gain in a subject comprising administering to the subject a composition comprising an effective amount of an extract mixture comprising a whole grape extract and at least one of a honeybush extract and a grape seed extract to effectively treat, prevent, or manage weight gain in the subject.

29. A method of reducing fat absorption, transport, deposition, production and secretion in a subject comprising administering to the subject a composition comprising an effective amount of an extract mixture comprising a whole grape extract and at least one of a honeybush extract and a grape seed extract to effectively reduce fat absorption, transport, deposition, production and secretion in the subject.

30. A method of facilitating body weight lost in a subject comprising administering to the subject a composition comprising an effective amount of an extract mixture comprising a whole grape extract and at least one of a honeybush extract and a grape seed extract to effectively facilitate weight loss in the subject.

31. A method of maintaining body weight of a subject comprising administering to the subject a composition comprising an effective amount of an extract mixture comprising a whole grape extract and at least one of a honeybush extract and a grape seed extract to effectively maintain body weight of the subject.

32. A method for treating, preventing or managing skin conditions or disorders associated with skin oil production in a subject comprising administering to the subject a composition comprising an effective amount of an extract mixture comprising a whole grape extract and at least one of a honeybush extract and a grape seed extract to effectively treat, prevent or manage skin conditions or disorders associated with skin oil production in the subject.

33. A method of synergistically inhibiting DGAT-I in a subject comprising administering to the subject a composition comprising an effective amount of an extract mixture comprising a whole grape extract and at least one of a honeybush extract and a grape seed extract to synergistically inhibit DGAT-I in the subject.
